(19) United States
(12) Patent Application Publication
(10) Pub. No.: US 2011/0245885 A1
(43) Pub. Date: Oct. 6, 2011

(54) Protection Sleeve Retention Device

(76) Inventor: Sean Powell, Coatesville, PA (US)

(21) Appl. No.: 12/899,241

(22) Filed: Oct. 6, 2010

Related U.S. Application Data
(60) Provisional application No. 61/251,935, filed on Oct. 15, 2009.

(51) Int. Cl. A61B 17/56

(52) U.S. Cl. 606/86 R

(57) ABSTRACT

A system for inserting an implant into a bone comprises a base, a first arm coupled to and extending away from the base in a first direction, a distal end of the first arm configured to removably attach to an implant so that the base is in a desired orientation relative thereto. The system also comprises a second arm extending away from the base in alignment with a target structure of the implant and including a first aiming hole through which the target structure is to be accessed. The system also comprises a protection sleeve and a first aiming hole configured such that, in a first orientation, the protection sleeve is frictionally locked within the first aiming hole and, when rotated therewithin to a second configuration, the protection sleeve is free to move therethrough.

### PRIORITY CLAIM

The present application claims priority to U.S. Provisional Application Ser. No. 61/251,935 filed on Oct. 15, 2009 to Sean Powell, the entire disclosure of which is incorporated herein by reference.

### FIELD OF THE INVENTION

The present invention is related to the field of bone fixation and, more particularly, related to an aiming arm configured to guide a bone fixation element to a target portion of a bone and subsequently lock the bone fixation element to the bone.

### BACKGROUND

The fixation and stabilization of bones in living bodies commonly involves an implant (e.g., an intramedullary nail, etc.) inserted into a target bone. Mechanical aiming instruments are often used to aid in alignment of the implant with or within the target bone. Such an aiming arm generally comprises a protection sleeve inserted through a hole oriented towards the implant to maintain a desired position of the implant relative to the aiming arm and to provide a barrier to protect soft tissue from damage during implantation. Such protection sleeves usually also comprises an opening for the introduction of drills, screws and other instruments or implants therethrough. Presently available aiming arms provide a tensioning mechanism such as a spring, set-screw, or a spring-loaded contact element configured to tension an outer wall of the protection sleeve as it is inserted through the aiming arm. Specifically, these tensioning mechanisms apply a constant frictional force to the protection sleeve for the entire period of insertion through the aiming arm and against the implant or must be selectively engaged or disengaged by an alternate means.

### SUMMARY OF THE INVENTION

The present invention is directed to a system for inserting an implant into a bone, comprising a base defining an open central area sized to receive a portion of a patient's anatomy including a target bone into which an implant is to be inserted and a first arm coupled to the base and extending away therefrom in a first direction, a distal end of the first arm being configured to temporarily mount a proximal end of an implant thereto so that the base is in a desired orientation relative to the implant extending along a desired axis in a second direction opposite the first direction passing through the central area of the base in combination with a second arm separated laterally from the axis so that, when the first arm is in the first target orientation, the second extends away from the base substantially parallel to the axis in alignment with a target structure of an implant coupled to the first arm, the second arm including a first aiming hole through which the target structure is to be accessed and a protection sleeve sized for insertion through the first aiming hole through an intervening portion of soft tissue located adjacent to the bone and to the target structure of an implant coupled to the first arm, one of the protection sleeve and the first aiming hole being configured so that the protection sleeve is frictionally locked in position within the first aiming hole when the protection sleeve is rotated to a first orientation relative to the first aiming hole and, when rotated from the first orientation to a second orientation, the protection sleeve is free to move within the first aiming hole.

### BRIEF DESCRIPTION OF THE DRAWINGS

FIG. 1 shows a perspective view of an exemplary system according to the present invention;

FIG. 2 shows a zoomed partial cross-sectional view of the system of FIG. 1 in an unlocked position;

FIG. 3 shows a second zoomed partial cross-sectional view of the system of FIG. 1 in a locked position;

FIG. 4 shows another perspective view of the system of FIG. 1;

FIG. 5 shows another perspective view of the system of FIG. 1;

FIG. 6 shows a perspective view of a protection sleeve according to the present invention;

FIG. 7 shows a perspective view of a second exemplary system according to the present invention;

FIG. 8 shows a first zoomed partial cross-sectional view of the system of FIG. 7 in an unlocked position;

FIG. 9 shows a second zoomed partial cross-sectional view of the system of FIG. 7 in locked position;

FIG. 10 shows another perspective view of the system of FIG. 7; and

FIG. 11 shoes another perspective view of the system of FIG. 7.

### DETAILED DESCRIPTION

The present invention is directed to a system and method for the fixation of a bone in a living body. Specifically, the present invention is directed to an exemplary protection sleeve configured for non-frictional insertion via a hole extending through an aiming arm. After the protection sleeve has been inserted to a target position relative to an implant (e.g., an intramedullary nail) positioned by the aiming arm, the protection sleeve is rotated to increase a frictional engagement between outer walls thereof with the hole through the aiming arm. Specifically, the protection sleeve is substantially cylindrical except for two flattened diametrically opposing walls extending along a portion of a longitudinal length thereof so that a width of the protection sleeve extending between the flattened walls is smaller than a diameter of outlying portions thereof. The hole formed through the aiming arm comprises leaf spring walls that are radially expandable upon application of a sufficient force thereto. In a first position, the leaf spring walls assume a cross-sectional shape substantially similar to a cross-sectional shape of the protection sleeve with a length between the leaf springs being substantially equivalent to or greater than the width of the portion of the protection sleeve extending between the flattened walls. Upon rotation of the protection sleeve about a longitudinal axis thereof, the increased diameter portions of the protection sleeve come into engagement with the leaf springs applying radially outward force thereto radially expanding the leaf spring walls. Frictional engagement between the leaf spring walls and the increased diameter outer wall of the protection sleeve then helps to maintain a desired position of the protection sleeve relative to the aiming arm. An exemplary embodiment of the present invention thus permits a physician or other user to rotationally lock and unlock the protection sleeve from the aiming arm as needed for the completion of a target bone fixation procedure. As used in this application, the term proximal refers to a direction approaching an end of the system away from a target bone hole for the insertion of a bone implant and the term distal refers to a direction approaching or located within the target bone hole. In an operative configuration, the distal end of the exemplary protection sleeve according to the present invention is inserted into the bone hole and inserted to a target position within the bone.

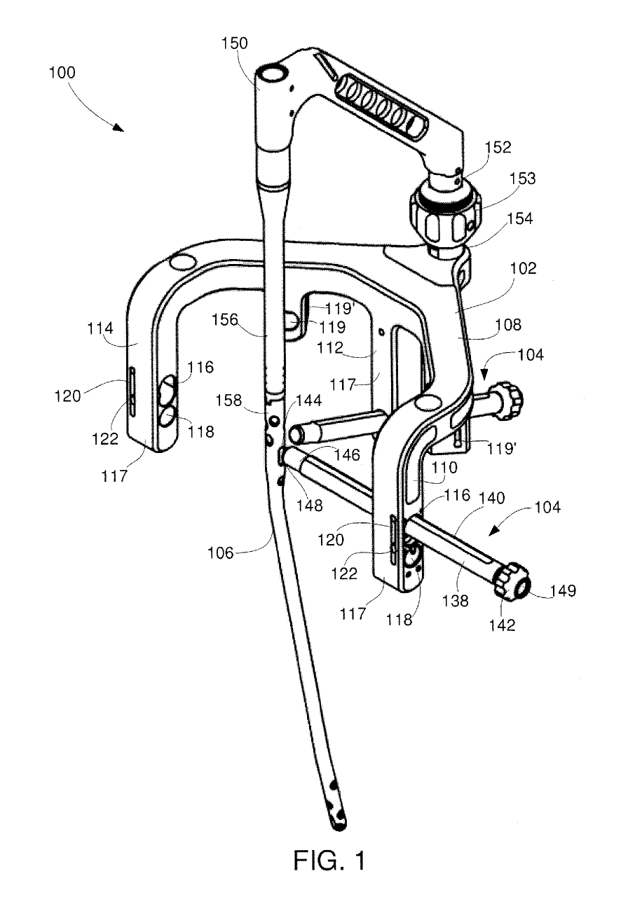

FIG. 1 shows a first exemplary system 100 according to the present invention. The system 100 comprises an aiming arm 102, a protection sleeve 104 and an intramedullary nail 106 configured for insertion into a bone (not shown) in accordance with an exemplary bone fixation procedure. The aiming arm 102 comprises a semi-circular element 108 comprising first, second third, fourth and fifth arms 110, 111, 112, 113, 114, extending distally therefrom. Each of the first, second, third, fourth and fifth arms 110, 111, 112, 113, 114 are spaced apart from one another along the semi-circular element 108, with the first and fifth aims 110, 114 located at ends of the semi-circular element 108 substantially diametrically opposed to one another. In an exemplary embodiment, the aiming arm 102 is formed of a radiolucent material (e.g., carbon fiber, plastic or aluminum). The first, second, third, fourth and fifth arms 110, 111, 112, 113, 114 extend perpendicular to a plane including the semi-circular element 108 and are substantially parallel to one another. Each of the first and fifth arms 110, 114 also comprises a combination hole 116 formed as two substantially circular holes located overlapping one another with the two circular holes open to one another, as those skilled in the art will understand, and a circular hole 118 located adjacent to the combination hole 116. A slot 120 extends into each of the combination holes 116 from a lateral wall 117 of the corresponding one of the first and fifth arms 110, 114 and extends through the combination hole 116 substantially perpendicular to axes of the holes 116, 118. A width of the slot 120 is smaller than the diameters of the circular holes making up the combination hole 116 and the circular hole 118 and a length of the slot 120 along the length of the respective one of the first and fifth arms 110, 114 is sufficient to intersect the entire length of the combination hole 116 and circular hole 118 in the same direction. The second, third and fourth arms 111, 112, 113 each only comprise a single hole 119 and a respective slot 119′ configured to receive the protection sleeve 104 therethrough, as will be described in greater detail later on.

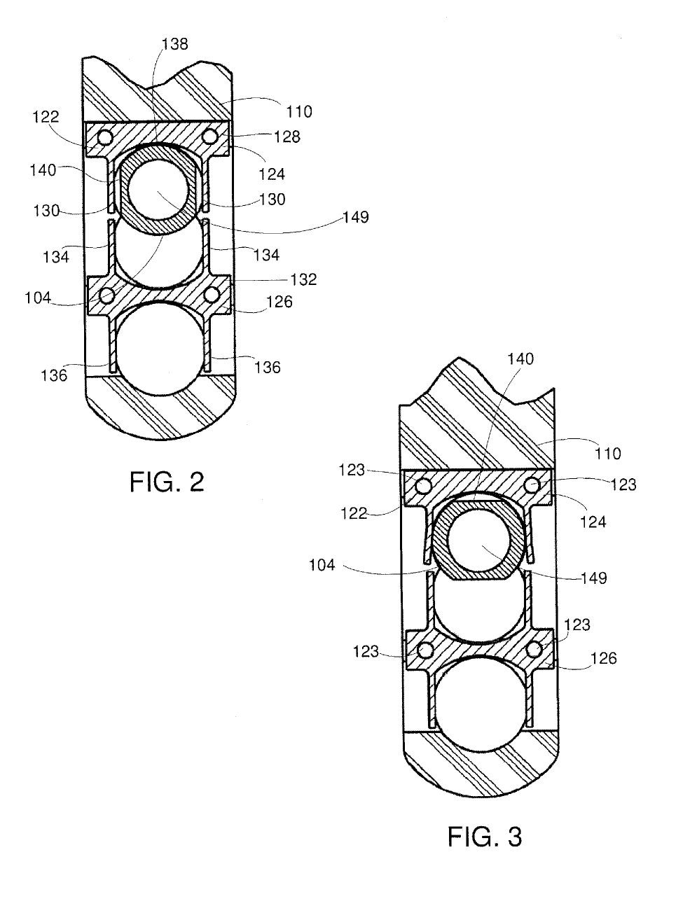

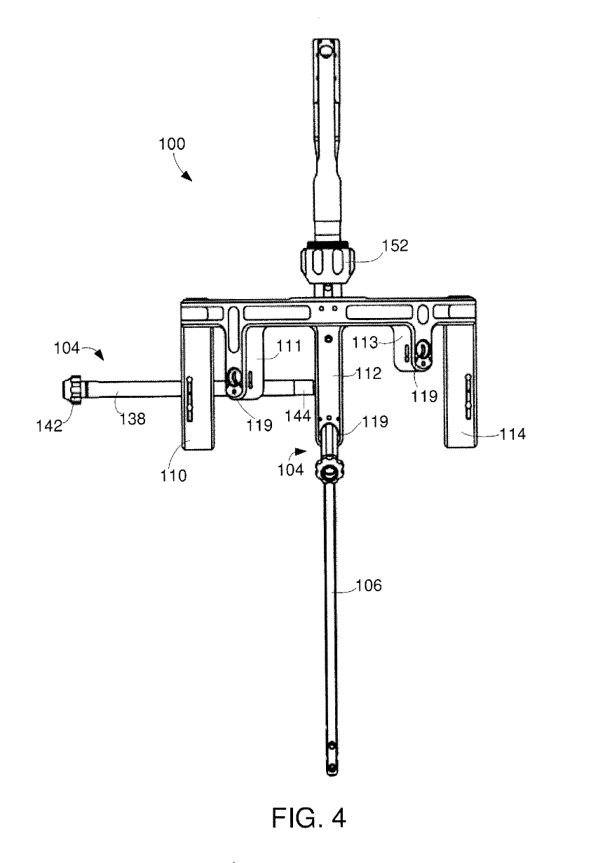

Spring elements 122 housed in each of the slots 120 comprise a first spring member 124 and a second spring member 126. The spring element 122 is formed of a sufficiently ductile material. The first and second spring members 124, 126 are not connected to one another and may optionally be employed alone if, for example, the single hole 119 is used in place of the combination hole 116. Specifically, the second, third and fourth arms 111, 112, 113 may comprise a spring element 122 positioned within the single holes 119 configured to apply a radially compressive force to any protection sleeve 104 inserted therethrough. The spring element 122 is held within the slot by one or more pins 123 forming one of a permanent and a removable connection. In an exemplary embodiment, the spring element may be formed of 302 Stainless Steel, 316 Stainless Steel, 17-7PH, Nitinol or Elgiloy®. The first spring member 124 includes a planar element 128 having first and second leaf springs 130 extending into a first circular portion of the combination hole 116, as shown in the partial cross-sectional views of FIGS. 2-3. The leaf springs 130 are biased to a position in which a width of the space therebetween is smaller than a diameter of a non-flattened portion of the protection sleeve 104 but greater than a width of a flattened portion thereof, as will be described in greater detail hereinafter. Similarly, the second spring member 126 is formed with a planar element 132 having a first set of leaf springs 134 extending therefrom into the combination hole 116 in a first direction and a second set of leaf springs 136 extending into the circular hole 118 in a second direction opposite the first direction. Each of the first and second sets of leaf springs 134, 136 is sized substantially similarly to the leaf springs 130 with a space formed between each pair of leaf springs being smaller than a diameter of the non-flattened portion of the protection sleeve 104 but greater than a width of the flattened portion thereof.

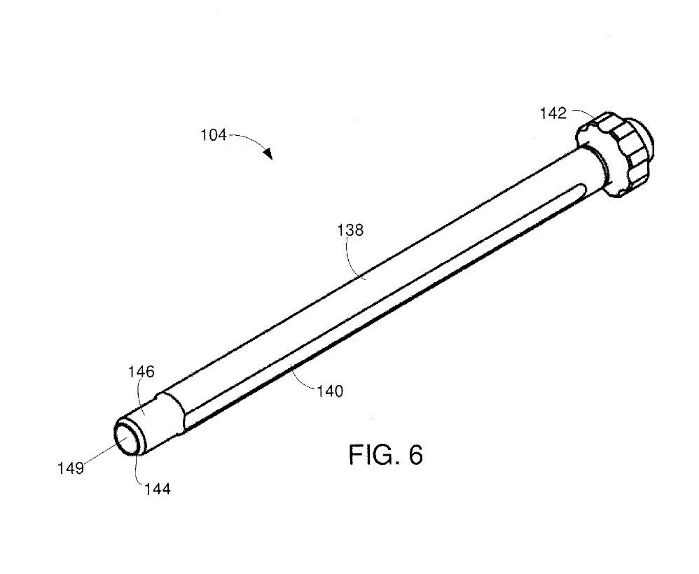

The protection sleeve 104 includes an elongated shaft 138 extending from an increased diameter head 142 with an externally scalloped shape, as those skilled in the art will understand, at a proximal end thereof to a distal end 144 having a reduced thickness portion 146. The reduced thickness portion is substantially cylindrical in shape and comprises a smaller diameter than the shaft 138, as shown in greater detail in FIG. 6. An outer surface of the shaft 138 includes a plurality of generally cylindrical portions and a plurality of flattened portions including flattened sides extending substantially parallel to one another and parallel to a longitudinal axis of the sleeve 104. Specifically, the protection sleeve 104 in this embodiment comprises two flat surfaces 140 formed on opposing diametrical sides of the shaft 138, each of the flat surfaces 140 extending along a predetermined length of the shaft, as shown in FIG. 1. The distal end 144 of the protection sleeve 104 is formed with a substantially circular cross-section having a diameter smaller than that of the shaft 138 to aid in insertion of the protection sleeve 104 into the bore 148 of the intramedullary nail 106. A channel 149 extends through the protection sleeve 104 and is open at proximal and distal ends thereof, the channel 149 having a substantially circular cross-section and being dimensioned to permit insertion of a medical instrument or implant therethrough.

The aiming arm 102 further comprises an insertion handle 150 including a first end extending away from the semi-circular element 108 substantially parallel to and opposite a direction of the arms 110-114. The insertion handle 150 is threadedly connected to a joint 152 and tightened therein via an adjusting knob 153 to lock a position thereof and prevent any movement of the insertion handle 150 relative to the semi-circular element 108. The insertion handle 150 extends from the joint 152 along a curved path to a second end 156 facing back toward a plane of the semi-circular element 108 and configured to engage a proximal end 158 of the intramedullary nail 106 so that the nail 106, when attached thereto, extends through the plane of the semi-circular element 108 with the bores 148 thereof aligned with the combination holes 116 and single holes 118 of the first and fifth arms 110, 114 or the single holes 119 of the second, third and fourth arms 111, 112, 113. Thus the aiming arm 102 and the insertion handle 150 hold the nail 106 in a desired position during insertion to facilitate implantation, as those skilled in the art will understand.

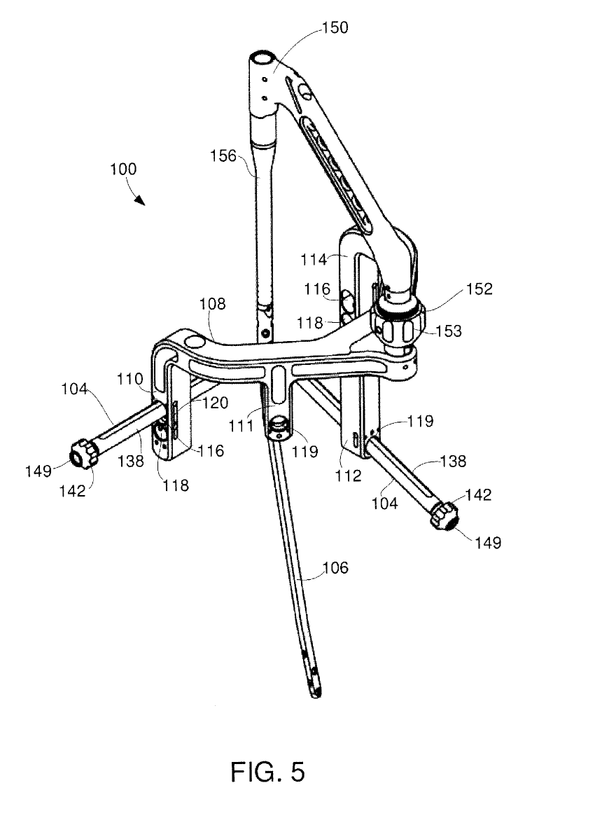

In accordance with an exemplary method of the present invention, a drill (not shown) is used to drill a first bore opening to the medullary canal of a bone (not shown) so that the intramedullary nail 106 may be inserted therein. The intramedullary nail 106 is then mounted to the second end 156 of the insertion handle 150 prior to insertion thereof into the bone. A user then determines which of the arms 110-114 will receive the protection sleeve 104 based on the position of a fracture in the bone or a pending pathological fracture and on the geometry of the nail 106. In the provided illustration, the first aim 110 is selected to receive the protection sleeve 104. Thus, the insertion handle 150 and the intramedullary nail 106 are oriented so that, when the intramedullary nail 106 is inserted to a desired position in the first bore (not shown), a drill inserted through the protection sleeve 104 and through the combination hole 116 or the circular hole 118 of the first arm 110 is in alignment with the bore 148 extending through the intramedullary nail 106. As shown in FIGS. 1 and 5, the intramedullary nail 106 comprises a plurality of additional bores 148′ extending therethrough at a plurality of angles, each of the bores 148′ being substantially perpendicular to a longitudinal axis of the intramedullary nail 106. Accordingly, a user of the system 100 may select more than one of the arms 110-114 to receive the protection sleeve 104 therethrough. Thus, the intramedullary nail 106 may receive any plurality of protection sleeves 104 without deviating from the scope of the present invention. The intramedullary nail 106 is then inserted into the bone and the protection sleeve 104 is inserted into the combination hole 116 in a first configuration with the flat surfaces 140 aligned with the leaf springs 130 so that the protective sleeve 104 slides therepast with a minimal amount of resistance. Once the protection sleeve 104 has been inserted to the target depth in the bore 148, the protection sleeve 104 is rotated by approximately 90 degrees so that the portions of the shaft 138 having an increased diameter relative to the flat surfaces 140 are in contact with the leaf springs 130 deflecting the leaf springs 130 radially outward against the spring bias and frictionally engaging the leaf springs 130 with the substantially cylindrical portions of the shaft 138 locking the protection sleeve 104 in the desired position. Once the protection sleeve 104 has been locked in this desired position, a drill, screw or other instrument or implant may be inserted through the channel 149 into the bore 148 to drill a transverse second bore at an angle to the first bore (not shown). As those skilled in the art will understand, the second bore extends through a lateral cortex of the bone at a point selected to align with the bore 148 when the nail 106 is in a desired position within the bone.

In the embodiment discussed above, the protection sleeve 104 is formed with a substantially circular cross-section having two flat surfaces 140 formed on opposing walls thereof. In a first alternate embodiment of the present invention, the shaft 138 may be formed with any cross-sectional shape, including, but not limited to square, hexagonal, octagonal, or another polygon shape so long as the cross-sectional geometry of the sleeve 104 relative to the leaf springs 130 such that, in a first orientation, a reduced diameter or reduced width aspect is presented to the leaf springs 130 and, in a second orientation, a larger diameter or width portion is presented to the leaf springs 130 deflecting the leaf springs 130 radially outward and increasing a resistance to the movement of the protection sleeve 104 relative to the one of the arms 110-114 through which it is inserted. The protection sleeve 104 may further comprise any type of recesses and is not limited to the flat surfaces 140. Specifically, the recesses may be concave, convex, helical or any other shape wherein an outer diameter of the recess is smaller than an outer diameter of the shaft 138. Furthermore, the protection sleeve 104 may comprise one or any plurality of recesses formed along an outer wall thereof. In yet another embodiment, the recesses may be replaced by features having the same outer diameter as the shaft 138 but which comprise a different material than the shaft 138 such as Nylon or Teflon or may comprise an alternate surface finish (e.g., knurling, grooving or threading) with properties selected to reduce or enhance frictional engagement of portions of the outer surface with the leaf springs 130 relative to the frictional engagement of a surface finish of other portions of the outer surface of the shaft 138.

In another embodiment of the present invention, the aiming arm 102 may comprise one or more leaf springs 130. Furthermore, the leaf springs 130 may be replaced with coil springs, thin flexible mechanical elements (e.g., belleville washers), wave spring, compressible cylinders, wave washers or portions of elastic material (e.g., rubber, plastic). The leaf springs 130 may be intrinsic with the arms 110-114 or, in an alternate embodiment, a separate element may be used to enhance contact therewith. The planar element 128 may be permanently affixed to the slot 120 by use of a pin or other mechanical attachment mechanism known in the art or, in an alternate embodiment, may be removable therefrom as needed.

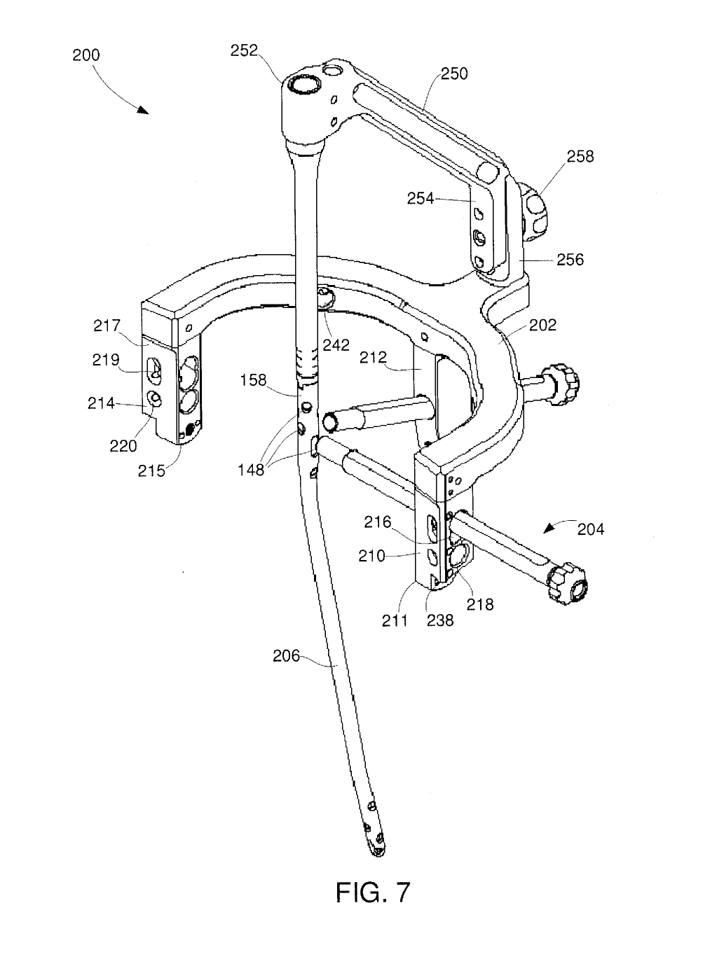

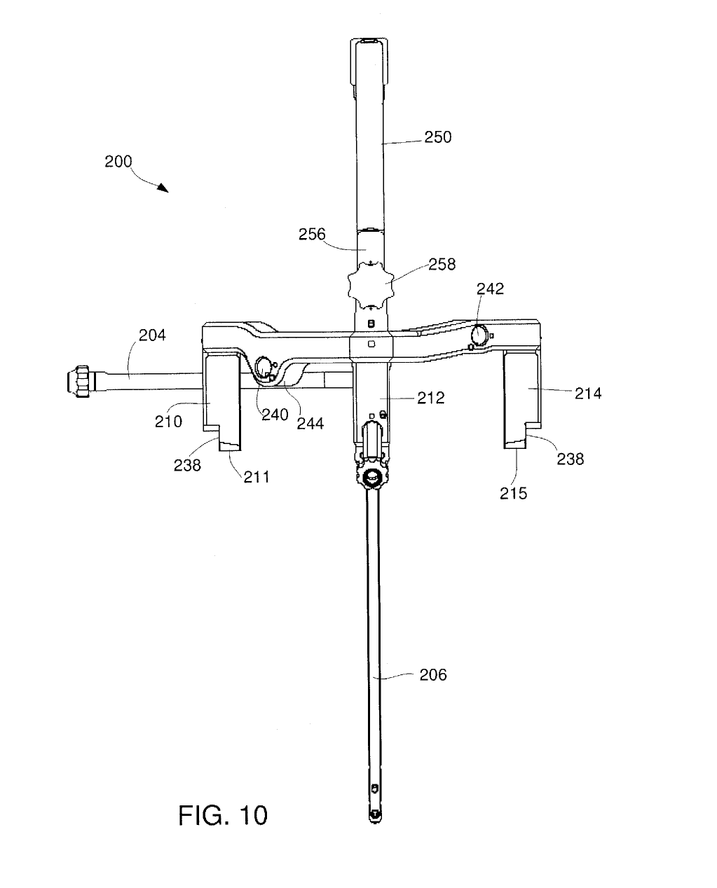

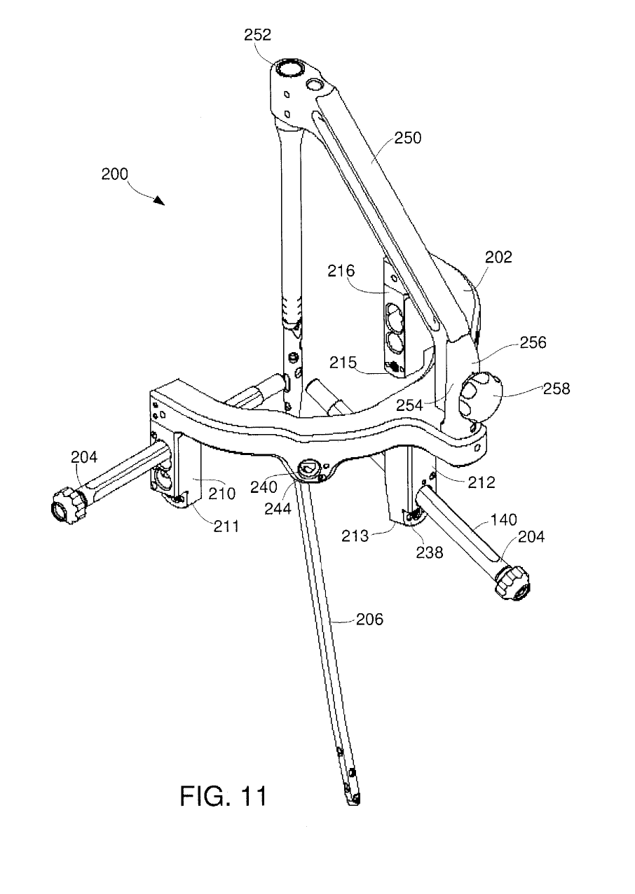

FIGS. 7-11 depict a system 200 according to another exemplary embodiment of the present invention. The system 200 is formed substantially similarly to the system 100, with like elements referenced by like reference numerals. The system 200 comprises an aiming arm 202, a protection sleeve 204 and an intramedullary nail 206 formed substantially similarly to the respective elements of system 100. A semi-circular element 208 of the aiming arm 202 comprises first, second and third arms 210, 212, 214 extending distally therefrom and spaced apart from one another. In an exemplary embodiment, the first, second and third arms 210, 212, 214 extend substantially perpendicularly from a plane housing the aiming arm 202 and are substantially parallel to one another. Similar to the system 100, each of the first, second and third arms 210, 212, 214 comprises a combination hole 216 extending laterally therethrough and a circular hole 218 adjacent thereto. Instead of a slot 120 as taught in system 100, the system 200 comprises a lateral, substantially oval hole 219 and a lateral circular hole 220 extending through a lateral wall 217 thereof. Each of the lateral holes 219, 220 have a length corresponding to the length of the combination hole 216 and circular hole 218, respectively. In an exemplary embodiment, the lateral oval hole 219 and the lateral circular hole 220 are configured and dimensioned so that spring elements 222, 224, 226 inserted therethrough project into at least a portion of the combination hole 216 and the circular hole 218, respectively, as will be described in greater detail hereinafter.

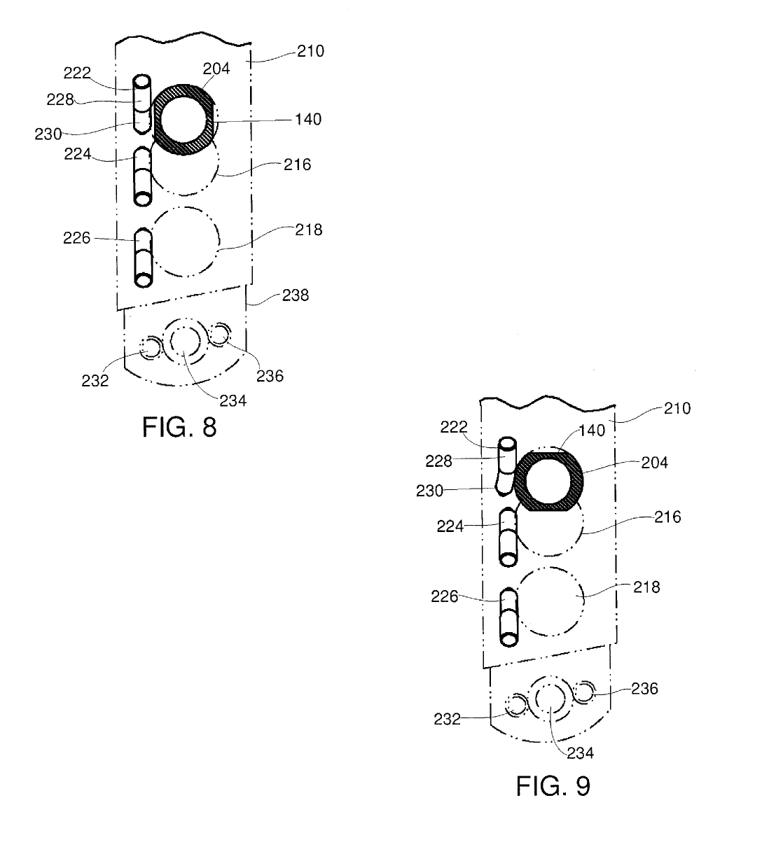

Exemplary spring elements 222, 224, 226 according to the exemplary embodiment are configured as substantially planar elements formed of a material substantially similar to a material of the spring elements 122 of system 100. Each of the spring elements 222, 224 and 226 is separate from the others and is configured for separate, permanent insertion into one of the lateral holes 219, 220. The spring elements 222, 224, 226 are held in place within respective ones of the lateral holes 219, 220 by a friction fit (e.g., during manufacturing). Each of the spring elements 222, 224, 226 comprises a first portion 228 and a second portion 230, the second portion 230 being configured and dimensioned to deflect laterally away from a center of the combination hole 216 or circular hole 218 upon application of a pressure thereto (e.g., by rotation of the protection sleeve 204), as described in greater detail with respect to the system 100. Each of the spring elements 222, 224, 226 is positioned to project partially into the combination hole 216 and circular hole 218 by a length substantially equivalent to a depth of a cutout formed in the protection sleeve forming the flattened portion 140, as also described in greater detail earlier. This configuration permits the spring elements to remain substantially unobstructed when the protection sleeve 204 is inserted into the combination hole 216 in the configuration shown in FIG. 8. Upon rotation of the protection sleeve 204 to the position shown in FIG. 9, a radially expansive force is applied to the spring element 222 causing the second portion 230 to deflect radially outward.

As shown in FIGS. 8 and 9, each of the first, second and third arms 210, 212, 214 also comprises additional locking holes 232, 234, 236 adjacent respective ends 211, 213, 215 thereof. The additional locking holes 232, 234, 236 may be used to attached extension pieces (not shown) to the system 100. The ends 211, 213, 215 also comprise stepped portions 238 having a thickness reduced relative to proximal portions of the arms 210, 212, 214 to permit attachment to the extension pieces (not shown).

The exemplary embodiment of system 200 obviates the need for additional arms disposed between the first, second and third arms 210, 212, 214, instead replacing these arms with first and second sleeve holes 240, 242. Specifically, the first sleeve hole 240 is provided on a portion of the aiming arm 202 substantially equidistant from the first arm 210 and the second arm 214. A partially circular extension portion 244 of the aiming arm 202 extends distally from the aiming arm 202 and along a curve having a radius of curvature suited to the dimensions of the sleeve hole 240, as those skilled in the art will understand. In an exemplary embodiment, the position of the extension portion 244 and the sleeve hole 242 are selected so that a drill sleeve inserted through the sleeve hole 242 is aligned with an opening extending through the intramedullary nail 206. It is therefore noted that the dimensions of the extension portion 244 may be varied to suit the requirements of a predetermined procedure. Similarly, the sleeve hole 242 positioned between the second and third arms 212, 214 may be positioned elsewhere along the aiming arm to conform to the requirements of an intramedullary nail 206 to be used therewith. In the embodiment shown, a portion of the aiming arm 202 housing the sleeve hole 242 is longitudinally offset from the plane housing the aiming arm 202. It is respectfully submitted that the aiming arm 202 may be formed with any geometry to permit a drill sleeve 204 inserted therethrough to intersect with a target portion of the intramedullary nail 206 without deviating from the spirit and scope of the present invention.

The system 200 further comprises an insertion handle 250 formed substantially similarly to the insertion handle 150 of the system 100. Specifically, the insertion handle 250 extends away from the aiming arm 202 along a curved path in a direction substantially opposite a direction of the arms 210, 212, 214 to an end 252 facing back toward the plane of the aiming arm 202. A locking portion 254 of the insertion handle 250 is configured for locking engagement with a locking portion 256 of the aiming arm 202, by, for example, tightening of a threaded knob 258 through each of the locking portions 254, 256. Tightening of the knob 258 locks a position of the insertion handle 250 relative to the aiming arm 202. The end 252 is configured to engage the proximal end 158 of the intramedullary nail 206 so that the nail 106, when attached thereto, extends through the plane of the aiming arm 202 with bores 148 thereof aligned with at least one of the combination holes 216, circular holes 218 or sleeve holes 240, 242. Thus the aiming arm 202 and the insertion handle 250 hold the nail 206 in a desired position during insertion to facilitate implantation. The present invention has been described with respect to intramedullary nails for the fixation of long bones. It is noted however, that the exemplary system of the present invention may also be employed in securing protection sleeves in aiming arms for surgical bone plates or artificial joints. Furthermore, the exemplary system of the present invention may also be used to secure a cylindrical instrument known in the art (e.g., a drill sleeve) into a hollow portion of another cylindrical instrument known in the art (e.g., a protection sleeve).

Although the present invention has been described with reference to preferred embodiments, it is submitted that various modifications can be made to the exemplary system and method without departing from the spirit and scope of the invention.

What is claimed is:

1. A system for inserting an implant into a bone, comprising:
a base defining an open central area sized to receive a portion of a patient's anatomy including a target bone into which an implant is to be inserted;
a first arm coupled to the base and extending away therefrom in a first direction, a distal end of the first arm being configured to removably mount a proximal end of an implant thereto so that the base is in a desired orientation relative to the implant extending along a desired axis in a second direction opposite the first direction passing through the central area of the base;
a second arm separated laterally from the axis so that the second arm extends away from the base substantially parallel to the axis in alignment with a target structure of an implant coupled to the first arm, the second arm including a first aiming hole through which the target structure is to be accessed; and
a first protection sleeve sized for insertion through the first aiming hole through an intervening portion of soft tissue located adjacent to the target structure of an implant coupled to the first arm, one of the first protection sleeve and the first aiming hole being configured so that the first protection sleeve is frictionally locked in position within the first aiming hole when the first protection sleeve is rotated to a first orientation relative to the first aiming hole and, when rotated from the first orientation to a second orientation, the first protection sleeve is free to move within the first aiming hole.

2. The system of claim 1, wherein the first aiming hole includes a first spring element extending thereinto and the first protection sleeve includes a first reduced diameter portion which, when rotated to face the first spring element, places the first protection sleeve in the second orientation relative to the first aiming hole, and rotation of the first protection sleeve so that a second increased diameter portion thereof engages the first spring element defining the first orientation so that the first spring element locks the increased diameter portion of the first protection sleeve within the first aiming hole.

3. The system of claim 2, wherein the first and second portions of the first protection sleeve are separated from one another around a circumference of the first protection sleeve.

4. The system of claim 1, wherein the first protection sleeve includes a first portion including surface features increasing a frictional engagement with an inner wall of the first aiming hole in comparison with a frictional engagement between a second portion of the first protection sleeve including a surface adapted to minimize frictional engagement with the inner wall of the first aiming hole.

5. The system of claim 4, wherein the first and second portions of the first protection sleeve are separated from one another around a circumference of the first protection sleeve.

6. The system of claim 1, wherein the base is substantially C-shaped with the first arm mounted at a central portion thereof and the second arm mounted at an end thereof.

7. The system of claim 1, further comprising a third arm separated laterally from the axis on a side of the open central area of the base opposite the second arm, the third arm being positioned to extend away from the base substantially parallel to the axis in alignment with a target structure of an implant coupled to the first arm, the third arm including a second aiming hole through which the target structure is to be accessed.

8. The system of claim 7, wherein the second hole is a combination hole.

9. The system of claim 2, wherein the first spring element is permanently affixed to the first arm.

10. The system of claim 2, wherein the first spring element is removably affixed to the first arm.

11. The system of claim 2, wherein the first spring element is one of a leaf spring, coil spring, belleville washer, compressible cylinder, wave spring and wave washer.

12. The system of claim 7, further comprising a fourth arm located between the second arm and the third arm, the fourth arm extending substantially parallel to the axis in alignment with the target structure of the implant and including a third aiming hole through which the target structure is to be accessed, the third aiming hole comprising a substantially circular cross-section.

13. The system of claim 12, further comprising a second protection sleeve sized for insertion through a selected one of the second and third aiming holes through an intervening portion of soft tissue located adjacent to a second target structure of the implant coupled to the first arm, one of the second protection sleeve and the selected one of the second and third aiming holes being configured so that the second protection sleeve is frictionally locked in position therewithin when the second protection sleeve is rotated to a third orientation relative to the selected aiming hole and, when rotated from the third orientation to a fourth orientation, the second protection sleeve is free to move within the selected aiming hole.

14. An apparatus for treating a bone, comprising:
a base defining an open central area sized to receive a portion of a patient's anatomy including a target bone into which an implant is to be inserted;
a first arm coupled to the base and extending away therefrom in a first direction, a distal end of the first arm being configured to temporarily mount a proximal end of an implant thereto so that the implant extends along a desired axis in a second direction opposite the first direction to pass through the central area of the base;
a second arm separated laterally from the axis so that the second arm extends away from the base substantially parallel to the axis in alignment with a target structure of an implant coupled to the first arm, the second arm including a first aiming hole through which the target structure is to be accessed, the first aiming hole being sized and oriented to receive therein a protection sleeve sized for insertion through the first aiming hole to an intervening portion of soft tissue located adjacent to the target structure of an implant coupled to the first arm, the first aiming hole being configured so that the protection sleeve is frictionally locked in position within the first aiming hole when the protection sleeve is rotated to a first orientation therein and so that, when rotated from the first orientation to a second orientation, the protection sleeve is free to move within the first aiming hole.

15. The apparatus of claim 14, wherein the first aiming hole includes a first spring element extending radially thereinto a distance selected so that a distance from a center of the first aiming hole to the first spring element is greater than a diameter of a reduced diameter portion of a protection sleeve to be inserted therethrough, the distance being less than a diameter of an increased diameter portion of the protection sleeve so that, when inserted into the first aiming hole and oriented so that the reduced diameter portion faces the first spring element, the protection sleeve is free to slide therethrough and, when inserted into the first aiming hole oriented so that the increased diameter portion faces the first spring element, the first spring element is deflected away from an unstressed configuration locking the protection sleeve within the first aiming hole.

16. A method for fixing an implant in a bone, comprising:
coupling to a proximal end of an implant to a first arm of an aiming device comprising a base so that a portion of the patient's anatomy within which the implant is to be fixed is received within an open central area of the base with the aiming device in a target orientation relative to the implant wherein the aiming device includes a second arm separated laterally from the axis so that, when the aiming device is in the target orientation, the second arm extends away from the base substantially parallel to the axis in alignment with a first target structure of the implant coupled to the first arm;
inserting through a first aiming hole in the second arm a first protection sleeve in a first rotational orientation within the first aiming hole through a portion of soft tissue located adjacent to the bone to engage the first target structure of the implant; and
after the first target structure has been engaged, rotating the first protection sleeve to a second orientation in which the first protection sleeve engages the first aiming hole to lock the first protection sleeve at a desired position within the first aiming hole.

17. The method of claim 16, wherein the first protection sleeve is rotated by approximately 90 degrees between the first and second orientations.

18. The method of claim 17, further comprising the step of inserting a medical instrument through the first protection sleeve to drill a bore into the bone at a desired angle so that the bore opens to the first target structure of the implant.

19. The method of claim 16, further comprising the step of inserting a second protection sleeve through a second aiming hole in a third arm of the aiming device in a third rotational orientation within the second hole through the soft tissue to engage a second target structure of the implant and, after the second target structure has been engaged, rotating the second protection sleeve to a fourth orientation in which the second protection sleeve engages the second aiming hole to lock the second protection sleeve at a desired position within the second aiming hole.
